(19) United States
(12) Patent Application Publication
(10) Pub. No.: US 2011/0124129 A1
(43) Pub. Date: May 26, 2011

(54) METHODS AND SYSTEMS TO CONTROL FLUID FLOW IN ACCORDANCE WITH A PREDETERMINED SEQUENCE

(75) Inventors: Thomas M. Zappia, Somerville, MA (US); Brandon T. Johnson, Cambridge, MA (US); Kate E. Christian, Somerville, MA (US)

(73) Assignee: BOSTON MICROFLUIDICS, Cambridge, MA (US)

(21) Appl. No.: 12/908,815

(22) Filed: Oct. 20, 2010

Related U.S. Application Data
(63) Continuation-in-part of application No. 12/228,081, filed on Jul. 16, 2008.

(51) Int. Cl. G01N 33/543

(52) U.S. Cl. 436/518; 422/68.1

(57) ABSTRACT

Methods and systems to perform sequential user-controlled fluidic assays, using substantially self-contained, portable, user-initiated fluidic assay systems, including user-initiated activation methods and systems.

### CROSS REFERENCE

This application is a continuation-in-part of U.S. Utility patent application Ser. No. 12/228,081, filed Jul. 16, 2008, and claims the benefit of:

U.S. Provisional Application No. 61/253,356, filed Oct. 20, 2009;

U.S. Provisional Application No. 61/253,365, filed Oct. 20, 2009;

U.S. Provisional Application No. 61/253,373, filed Oct. 20, 2009;

U.S. Provisional Application No. 61/253,377, filed Oct. 20, 2009;

U.S. Provisional Application No. 61/253,383, filed Oct. 20, 2009; and

U.S. Provisional Application No. 61/266,019, filed Dec. 2, 2009;

all of which are incorporated herein by reference in their entireties.

### TECHNICAL FIELD

Disclosed herein are methods and systems to perform sequential user-controlled fluidic assays, using substantially self-contained, portable, user-initiated fluidic assay systems.

### BACKGROUND

An immunoassay is a biochemical technique that can be used to detect the presence or absence of an infectious disease. Performing an immunoassay test involves several steps, all of which are usually automated in a lab by robotics. Over the past two decades, single-step disposable tests have been created. U.S. Pat. No. 4,943,522 is an example of using the lateral flow properties of a porous membrane to carry out an immunoassay process in a single step. Pregnancy and ovulation tests that require the application of urine is a good example of these types of tests.

While these lateral flow test only require a single application step, the application of each test step is dependent on the capillary flow of the sample to bring the reagents to the test area.

### SUMMARY

Disclosed herein are methods and systems to activate assay systems. An assay system may include a plurality of fluid chambers, fluid paths, a sample collection region, a test region, and a user-initiated and user-controlled activation system. Sequential portions of an assay may be activated by a one or more user-initiated actions, such as pressing a button one or more times.

Systems and methods disclosed herein may be implemented with respect to self-contained, point-of-care, portable, point-of-care, user-initiated fluidic assay systems.

Example assays include diagnostic assays and chemical detection assays. Diagnostic assays include, without limitation, enzyme-linked immuno-sorbent assays (ELISA), and may include one or more sexually transmitted disease (STD) diagnostic assays.

An example assay system includes a housing having one or more fluid chambers, a fluid controller system to dispense fluid from the one or more fluid chambers, and a user-initiated actuator to control the fluid controller system.

The actuator may be configured to serially move fluid controllers from functionally closed positions to functionally open positions, to control fluid flow from the fluid chambers.

The fluid controller system may be configured to dispense fluids serially, and may be configured to mix a plurality of fluids.

The housing may include an assay portion and the fluid controller system may be configured to dispense fluids from one or more of the fluid chambers to the assay portion.

The housing may include one or more fluid paths amongst the fluid chambers and/or to the assay portion, and the fluid controller system may be configured to serially align fluid chamber outlets with corresponding fluid paths.

The housing may include a sample chamber to receive an assay sample, such as a biological sample, and one or more of the fluid paths may include the sample chamber.

The user-initiated actuator system may include an external user-operated trigger mechanism to initiate the actuator system. The actuator system may include a mechanical actuator system, and may include a compressible spring actuator system.

The assay apparatus may include a display window to view assay results.

### BRIEF DESCRIPTION OF THE DRAWINGS/FIGURES

In the drawings, like reference numbers indicate identical or functionally similar elements. Additionally, the leftmost digit(s) of a reference number identifies the drawing in which the reference number first appears.

FIG. 1 is a process flowchart of a method of performing an assay with a substantially self-contained, point-of-care, user-initiated fluidic assay system.

FIG. 2 is a block diagram of a portable, point-of-care, user-initiated fluidic assay system.

FIG. 3 is a perspective view of another portable, point-of-care, user-initiated fluidic assay system.

FIG. 4 is a process flowchart of a method of preparing a portable, point-of-care, user-initiated fluidic assay system.

FIG. 5 is a process flowchart of a method of using an assay system prepared in accordance with FIG. 4.

FIG. 6 is a perspective view of an assay system 600, including a cover illustrated in a first position.

FIG. 7 is a cross-sectional view of assay system 600, including plungers 702, 704, and 706, wherein the cover is illustrated in the second position.

FIG. 8 is another cross-sectional view of assay system 600, wherein plungers 702, 704, and 706 are in corresponding initial or first positions.

FIG. 9 is another cross-sectional view of assay system 600, wherein plungers 702, 704, and 706 are in respective first intermediate positions.

FIG. 10 is another cross-sectional view of assay system 600, wherein plunger 704 is in a second position, and plungers 702 and 704 are in respective second intermediate positions.

FIG. 11 is another cross-sectional view of assay system 600, wherein plungers 702, 704 and 706 are in respective second positions.

FIG. 12 is an expanded cross-sectional view of a portion of assay system 600, including a portion of plunger 706 in the first position corresponding to FIG. 8.

FIG. 13 is another expanded cross-sectional view of a portion assay system 600, including a portion of plunger 706 in the intermediate position corresponding to FIG. 9.

FIG. 14 is another expanded cross-sectional view of a portion of assay system 600, including a portion of plunger 706 in the second position corresponding to FIGS. 10 and 11.

FIG. 15 is a cross-sectional perspective view of another assay system 1500.

FIG. 16 is a cross-sectional perspective view of another assay system 1600.

FIG. 17 is a profile view of another fluidic assay system.

FIG. 18 is a cross-sectional view of the fluidic assay system of FIG. 17.

FIG. 19 is a graphic depiction of an example pathway of an activation post of the fluidic assay system of FIG. 17.

FIG. 20 is a cross-sectional view of a housing portion of the fluidic assay system of FIG. 17, including the activation post pathway of FIG. 19 formed within an inner surface the housing portion.

FIGS. 21A through 21F graphically depict an example sequence of movements within the fluidic assay system of FIG. 17 in response to the activation post pathway of FIG. 20.

FIGS. 22A and 22B are cross-sectional views of an example fluid chamber of the fluidic assay system of FIG. 17.

In the drawings, the leftmost digit(s) of a reference number may identify the drawing in which the reference number first appears.

### DETAILED DESCRIPTION

An immunoassay is a biochemical test to detect a substance, or measure a concentration of a substance, in a biological sample such as blood, saliva, or urine, using a reaction between an antibody and an antigen specific to the antibody.

An immunoassay may be used to detect the presence of an antigen or an antibody. For example, when detecting an infection, the presence of an antibody against the pathogen may be measured. When detecting hormones such as insulin, the insulin may be used as the antigen.

Accordingly, where a method or system is described herein to detect a primary binding pair molecule using a corresponding second binding pair molecule, it should be understood that the primary binding pair molecule may be an antibody or an antigen, and the second binding pair molecule may be a corresponding antigen or antibody, respectively. Similarly, where a method or system is described herein to detect an antibody or antigen, the method or system may be implemented to detect a corresponding antigen or antibody, respectively.

Immunoassays may also be used to detect potential food allergens and chemicals, or drugs.

Immunoassays include labeled immunoassays to provide a visual indication of a binding pair of molecules. Labeling may include an enzyme, radioisotopes, magnetic labels, fluorescence, agglutination, nephelometry, turbidimetry and western blot.

Labeled immunoassays include competitive and non-competitive immunoassays. In a competitive immunoassay, an antigen in a sample competes with labeled antigen to bind with antibodies. The amount of labeled antigen bound to the antibody site is inversely proportional to the concentration of antigen in the sample. In noncompetitive immunoassays, also referred to as sandwich assays, antigen in a sample is bound to an antibody site. The labeled antibody is then bound to the antigen. The amount of labeled antibody on the site is directly proportional to the concentration of the antigen in the sample.

Labeled immunoassays include enzyme-linked immuno-sorbent assays (ELISA).

In an example immunoassay, a biological sample is tested for a presence of a primary binding pair molecule. A corresponding binding pair molecule that is specific to the primary binding pair molecule is immobilized on an assay substrate. The biological sample is contacted to the assay substrate. Any primary binding pair molecules in the biological sample attach to, or are captured by the corresponding binding pair molecules. The primary binding pair molecules are also contacted with labeled secondary binding pair molecules that attach to the primary binding pair molecules. This may be performed subsequent to, prior to, or simultaneously with the contacting of the primary binding pair molecule with the corresponding immobilized binding pair molecule. Un-reacted components of the biological sample and fluids may be removed, or washed from the assay substrate. Presence of the label on the assay substrate indicates the presence of the primary binding pair molecule in the biological sample.

The label may include a directly detectable label, which may be visible to a human observer, such as gold particles in a colloid or solution, commonly referred to as colloidal gold.

The label may include an indirect label, such an enzyme whereby the enzyme works on a substrate to produce a detectable reaction product. For example, an enzyme may attach to the primary binding pair molecule, and a substance that the enzyme converts to a detectable signal, such as a fluorescence signal, is contacted to the assay substrate. When light is directed at the assay substrate, any binding pair molecule complexes will fluoresce so that the presence of the primary binding pair molecule is observable.

An immunoassay may utilize one or more fluid solutions, which may include a dilutent solution to fluidize the biological sample, a conjugate solution having the labeled secondary binding pair molecules, and one or more wash solutions. The biological sample and fluids may be brought into contact, concurrently or sequentially with the assay substrate. The assay substrate may include an assay surface or an assay membrane, prepared with a coating of the corresponding binding pair molecules.

As described above, the second binding pair molecules may include an antigen that is specific to an antibody to be detected in a biological sample, or may include antibody that is specific to an antigen to be detected in the biological sample. By way of illustration, if the primary binding pair molecule to be detected is an antigen, the immobilized binding pair molecule and the secondary labeled binding pair molecule will be antibodies, both of which react with the antigen. When the antigen is present in the biological sample, the antigen will be immobilized by the immobilized antibody and labeled by the labeled secondary antibody, to form a sandwich-like construction, or complex.

It is known that non-specific or un-reacted components may be beneficially removed using wash solutions, often between processes and/or prior to a label detection process, in order to improve sensitivity and signal-to-noise ratios of the assay. Other permutations are possible as well. For example, a conjugate solution, such as a labeled secondary binding pair molecule solution may be mixed with or act as a sample dilutent to advantageously transport the biological sample to the assay substrate, to permit simultaneous binding of the primary binding pair molecule and the labeled secondary binding pair molecule to the immobilized binding pair molecule. Alternatively, or additionally, the sample dilutent may include one or more detergents and/or lysing agents to advantageously reduce deleterious effects of other components of the biological sample such as cellular membranes, non-useful cells like erythrocytes and the like.

Those skilled in the art will readily recognize that such fluid components and the order of the reactionary steps may be readily adjusted along with concentrations of the respective components in order to optimize detection or distinguishment of analytes, increase sensitivity, reduce non-specific reactions, and improve signal to noise ratios.

As will be readily understood, if the secondary antibody is labeled with an enzyme instead of a fluorescent or other immediately detectable label, an additional substrate may be utilized to allow the enzyme to produce a reaction product which will be advantageously detectable. An advantage of using an enzyme based label is that the detectable signal may increase over time as the enzyme works on an excess of substrate to produce a detectable product.

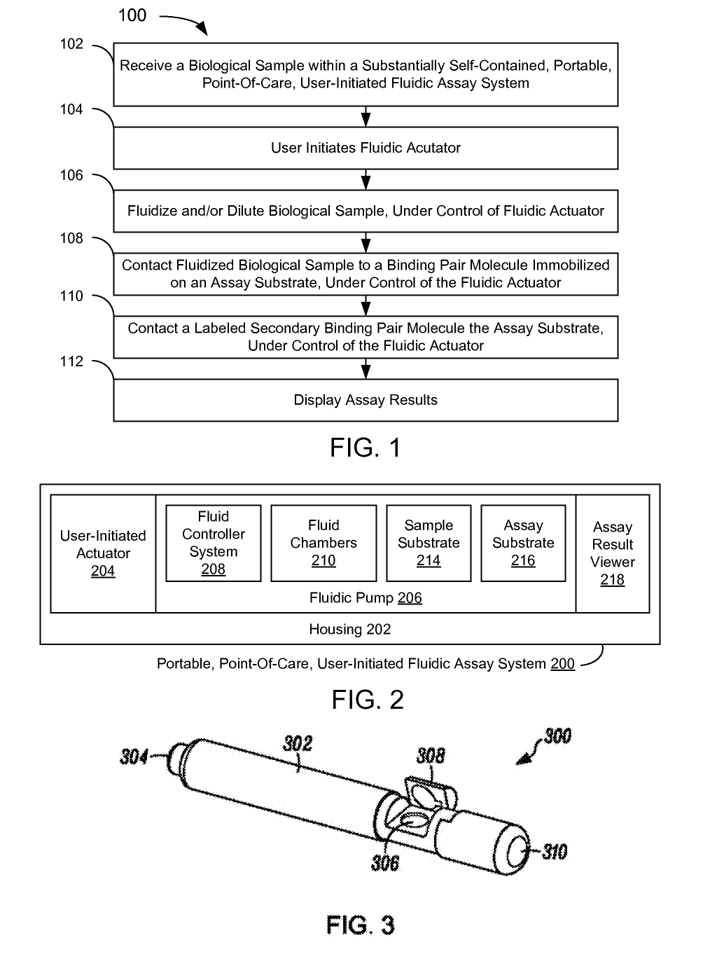

FIG. 1 is a process flowchart of a method 100 of detecting a primary binding pair molecule in a biological sample, using a substantially self-contained, point-of-care, user-initiated fluidic assay system. The primary binding pair molecule may correspond to an antibody or an antigen.

At 102, a biological sample is provided to the assay system. The biological sample may include one or more of a blood sample, a saliva sample, and a urine sample. The biological sample may be applied to a sample substrate within the assay system.

At 104, a fluidic actuator within the assay system is initiated by a user. The fluidic actuator may include a mechanical actuator, such as a compressed spring actuator, and may be initiated with a button, switch, or lever. The fluidic actuator may be configured to impart one or more of a physical force, pressure, centripetal force, gas pressure, gravitational force, and combinations thereof, on a fluid controller system within the assay system.

At 106, the biological sample is fluidized with a dilutent fluid. The dilutent fluid may flow over or through the sample substrate, under control of the fluid controller system.

At 108, the fluidized biological sample is contacted to a corresponding binding pair molecule that is specific to primary binding pair molecule. The corresponding binding pair molecule may be immobilized on an assay substrate within the assay system. The fluidized biological sample may flow over or through the assay substrate, under control of the fluid controller system.

Where the fluidized biological sample includes the primary binding pair molecule, the primary binding pair molecule attaches to the corresponding binding pair molecule and becomes immobilized on the assay substrate. For example, where the second binding pair molecule includes a portion of a pathogen, and where the biological sample includes an antibody to the pathogen, the antibody attaches to the antigen immobilized at the assay substrate.

At 110, a labeled conjugate solution is contacted to the assay substrate, under control of the fluid controller system. The labeled conjugate solution includes a secondary binding pair molecule to bind with the primary binding pair molecule. Where the primary binding pair molecule is immobilized on the assay substrate with the corresponding binding pair molecule, the secondary binding pair molecule attaches to the immobilized primary binding pair molecule, effectively creating a sandwich-like construct of the primary binding pair molecule, the corresponding binding pair molecule, and the labeled secondary binding pair molecule.

The secondary binding pair molecule may be selected as one that targets one or more proteins commonly found in the biological sample. For example, where the biological sample includes a human blood sample, the secondary binding pair molecule may include an antibody generated by a non-human animal in response to the one or more proteins commonly found in human blood.

The secondary binding pair molecule may be labeled with human-visible particles, such as a gold colloid, or suspension of gold particles in a fluid such as water. Alternatively, or additionally, the secondary binding pair molecule may be labeled with a fluorescent probe.

Where the labeled secondary binding pair molecule attaches to a primary binding pair molecule that is attached to a corresponding binding pair molecule, at 110, the label is viewable by the user at 112.

Method 100 may be implemented to perform multiple diagnostic assays in an assay system. For example, a plurality of antigens, each specific to a different antibody, may be immobilized on one or more assay substrates within an assay system. Similarly, a plurality of antibodies, each specific to a different antigen, may be immobilized on one or more assay substrates within an assay system

FIG. 2 is a block diagram of a portable, point-of-care, user-initiated fluidic assay system 200, including a housing 202, a user-initiated actuator 204, a fluidic pump 206, and an assay result viewer 218.

Pump 206 includes one or more fluid chambers 210, to contain fluids to be used in an assay. One or more of fluid chambers 210 may have, without limitation, a volume in a range of 0.5 to 2 milliliters.

Pump 206 includes a sample substrate 214 to hold a sample. Sample substrate 214 may include a surface or a membrane positioned within a cavity or a chamber of housing 202, to receive one or more samples, as described above.

Sample substrate 214 may include a porous and/or absorptive material, which may be configured to absorb a volume of liquid in a range of 10 to 500 μL, including within a range of up to 200 μL, and including a range of approximately 25 to 50 μL.

Pump 206 includes an assay substrate 216 to hold an assay material. Assay substrate 216 may include a surface or a membrane positioned within a cavity or chamber of housing 202, to receive one or more assay compounds or biological components, such as an antigen or an antibody, as described above.

Fluid chambers 210 may include a waste fluid chamber.

Pump 206 further includes a fluid controller system 208, which may include a plurality of fluid controllers, to control fluid flow from one or more fluid chambers 212 to one or more of sample substrate 214 and assay substrate 216, responsive to actuator 204.

Actuator 204 may include a mechanical actuator, which may include a compressed or compressible spring actuator, and may include a button, switch, lever, twist-activator, or other user-initiated feature.

Assay result viewer 218 may include a display window disposed over an opening through housing 202, over assay substrate 216.

FIG. 3 is a perspective view of a portable, point-of-care, user-initiated fluidic assay system 300, including a housing 302, a user-initiated actuator button 304, a sample substrate 306, and a sample substrate cover 308. Sample substrate cover 308 may be hingedly coupled to housing 302.

Assay system 300 further includes an assay result viewer 310, which may be disposed over an assay substrate. Assay result view 310 may be disposed at an end of assay system 300, as illustrated in FIG. 3, or along a side of assay system 300.

Assay system 300 may have, without limitation, a length in a range of 5 to 8 centimeters and a width of approximately 1 centimeter. Assay system 300 may have a substantially cylindrical shape, as illustrated in FIG. 3, or other shape.

Assay system 300, or portions thereof, may be implemented with one or more substantially rigid materials, and/or with one or more flexible or pliable materials, including, without limitation, polypropylene.

Example portable, point-of-care, user-initiated fluidic assay systems are disclosed further below.

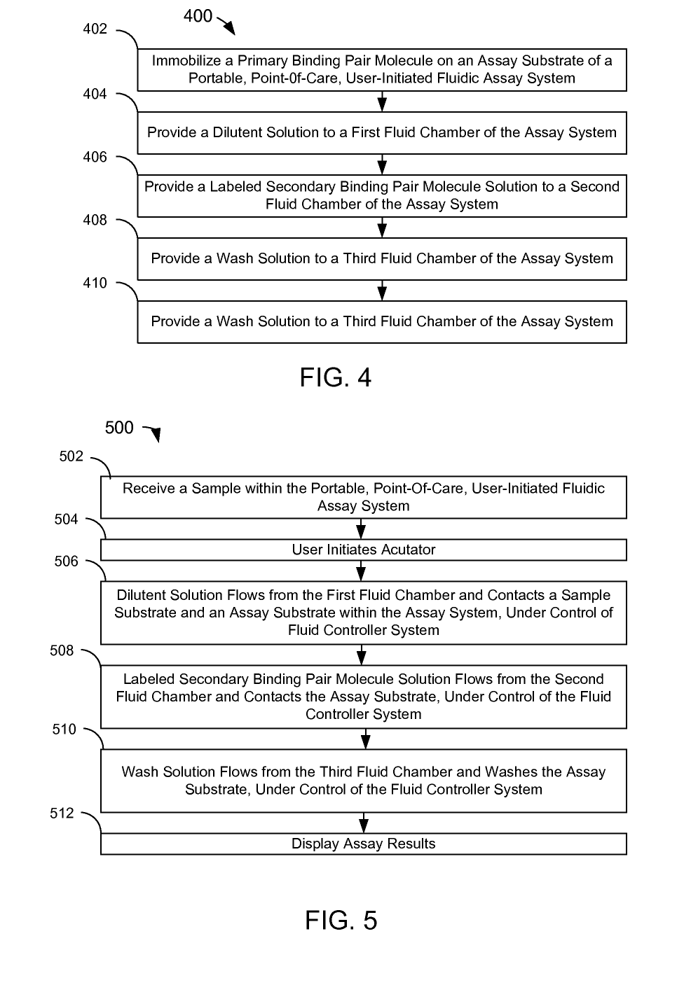

FIG. 4 is a process flowchart of a method 400 of preparing a portable, point-of-care, user-initiated fluidic assay system. Method 400 is described below with reference to assay system 200 in FIG. 2, for illustrative purposes. Method 400 is not, however, limited to the example of FIG. 2.

At 402, a binding pair molecule is immobilized on an assay substrate, such as assay substrate 216 in FIG. 2. The binding pair molecule may include an antigen specific to an antibody, or an antibody specific to an antigen.

At 404, a first one of fluid chambers 210 is provided with a dilutent solution to fluidize a sample.

At 406, a second one of fluid chambers 210 is provided with a labeled secondary binding pair molecule solution.

At 408, a third one of fluid chambers 210 is provided with a wash solution, which may include one or more of a saline solution and a detergent. The wash solution may be substantially similar to the dilutent solution.

FIG. 5 is a process flowchart of a method 500 of using an assay system prepared in accordance with method 400. Method 500 is described below with reference to assay system 200 in FIG. 2, and assay system 300 in FIG. 3, for illustrative purposes. Method 500 is not, however, limited to the examples of FIG. 2 and FIG. 3.

At 502, a sample is provided to a sample substrate, such as sample substrate 214 in FIG. 2, and sample substrate 306 in FIG. 3.

At 504, a user-initiated actuator is initiated by the user, such as user-initiated activator 204 in FIG. 2, and button 304 in FIG. 3. The user initiated actuator acts upon a fluid controller system, such as fluid controller system 208 in FIG. 2.

At 506, the dilutent solution flows from first fluid chamber and contacts the sample substrate and the assay substrate, under control of the fluid controller system.

As the dilutent fluid flows over or through the sample substrate, the sample is dislodged from the sample substrate and flows with the dilutent solution to the assay substrate.

At 508, the labeled secondary binding pair solution flows from the second fluid chamber and contacts the assay substrate, under control of the fluid controller system. The labeled secondary binding pair solution may flow directly to the assay substrate or may flow over or through the sample substrate.

At 510, the wash solution flows from the third fluid chamber and washes the assay substrate, under control of fluid controller system 208. The wash solution may flow from the assay substrate to a waste fluid chamber,

At 512, assay results are viewable, such as at assay result viewer 218 in FIG. 2, and assay result viewer 310 in FIG. 3.

An assay substrate may include a nitrocellulose-based membrane, available from Invitrogen Corporation, of Carlsbad, Calif.

Preparation of a nitrocellulose-based membrane may include incubation for approximately thirty (30) minutes in a solution of 0.2 mg/mL protein A, available from Sigma-Aldrich Corporation, of St. Louis, Mo., in a phosphate buffered saline solution (PBS), and then dried at approximately 37° for approximately fifteen (15) minutes. 1 μL of PBS may be added to the dry membrane and allowed to dry at room temperature. Alternatively, 1 μL of an N-Hydroxysuccinimide (NHS) solution, available from Sigma-Aldrich Corporation, of St. Louis, Mo., may be added to the dry membrane and allowed to dry at room temperature.

An assay method and/or system may utilize or include approximately 100 μL of PBS/0.05% Tween wash buffer, available from Sigma-Aldrich Corporation, of St. Louis, Mo., and may utilize or include approximately 100 μL of protein G colloidal gold, available from Pierce Corporation, of Rockland, Ill.

An assay method and/or system may be configured to test for Chlamydia, and may utilize or include a sample membrane treated with wheat germ agglutinin, to which an approximately 50 μL blood sample is applied. Approximately 150 μL of a lysing solution may then be passed through the sample membrane and then contacted to an assay substrate. Thereafter, approximately 100 μL of a colloidal gold solution may be contacted to the assay substrate. Thereafter, approximately 500 μL of a wash solution, which may include the lysing solution, may be contacted to the assay membrane without passing through the sample membrane.

Additional example assay features and embodiments are disclosed below. Based on the description herein, one skilled in the relevant art(s) will understand that features and embodiments described herein may be practiced in various combinations with one another.

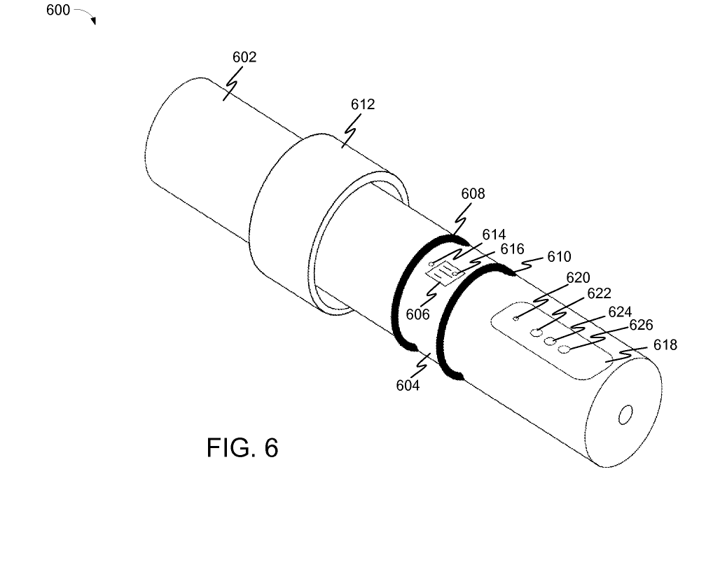

FIG. 6 is a perspective view of an assay system 600, including a body 602 having a sample collection region 604 to receive a sample collection pad or membrane 606, which may include a porous material such as, for example, a glass fiber pad, to absorb a fluid sample.

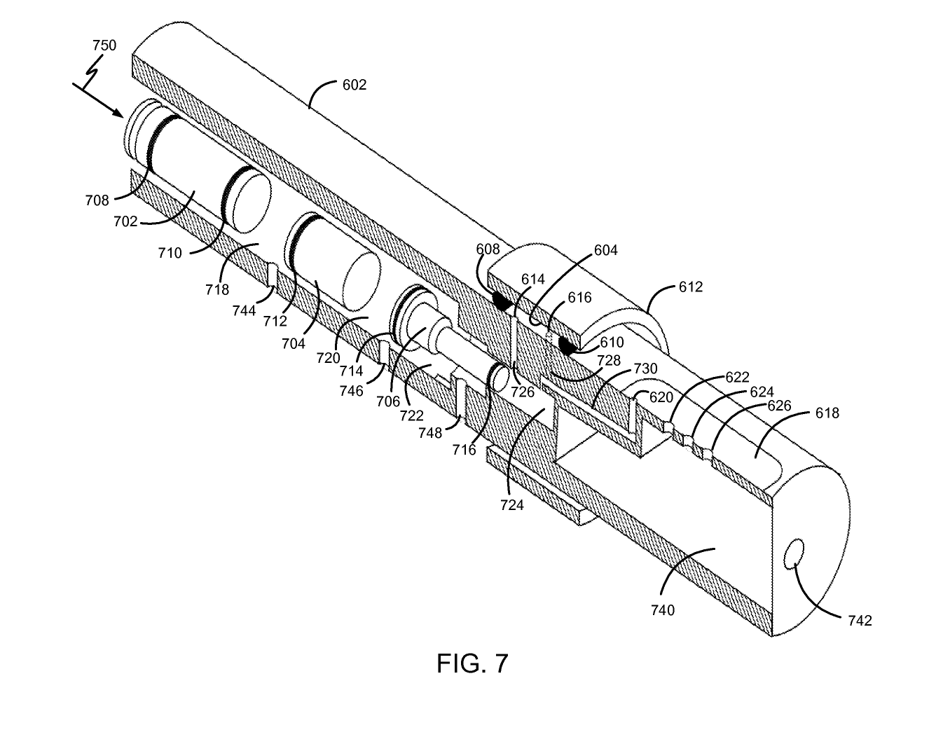

In the example of FIG. 6, sample collection region 604 is positioned between first and second O-rings 608 and 610, and system 600 includes a cover 612 slideably moveable relative to body 602, between a first position illustrated in FIG. 6, and a second position described below with reference to FIG. 7.

FIG. 7 is a cross-sectional view of assay system 600, wherein cover 612 is illustrated in the second position, and sample collection region 604 is bounded by an outer surface of body 602, an inner-surface of cover 612, and O-rings 608 and 610. O-rings 608 and 610 may provide a hermetic seal between sample collection region 604 and an external environment. When cover 612 is in the second position, sample collection region 604 may be referred to as a sample collection chamber.

In FIG. 6, sample collection region 604 includes openings 614 and 616 through the surface of body 602 associated with fluid passages within body 602. Opening 614 may be positioned adjacent to sample collection pad 606, and opening 616 may be positioned beneath sample collection pad 606. System 600 may be configured to provide a fluid through opening 614 into sample collection region 604 and to receive the fluid from sample collection region 604 through opening 616, to cause the fluid to pass through sample collection pad 606.

Body 602 may include an assay region 618 formed or etched within the surface of body 602, having an opening 620 through the surface of body 602 to receive fluid from an associated fluid passage. Assay region 618 may include one or more additional openings to corresponding fluid passages within body 602, illustrated here as openings 622, 624, and 626, to permit the fluid to exit assay region 618.

Assay region 618 may be configured to receive a test membrane having one or more reactive areas, each reactive area positioned on the test membrane in alignment with a corresponding one of openings 622, 624, and 626.

System 600 may include a substantially transparent cover to enclose assay region 618, such as to permit viewing of the test membrane, or portions thereof. The cover may include one or more fluid channels to direct fluid from opening 620 to the membrane areas aligned with openings 622, 624, and 626. Where system 600 includes a cover over assay region 618, assay region 618 may be referred to as an assay chamber.

In FIG. 7, system 600 includes plungers 702, 704, and 706. Plunger 706 is illustrated here as a multi-diameter or stepped plunger. Plunger 702 includes O-rings 708 and 710. Plunger 704 includes an O-ring 712. Plunger 706 includes O-rings 714 and 716. O-rings 708, 710, 712, 714, and 716 may be sized to engage corresponding inner surface portions of body 602. Plungers 702, 704, and 706 are each moveable within body 602 between respective first and second positions and, together with the inner surfaces of body 602, define fluid chambers 718, 720, 722, and 724.

In the example of FIG. 7, body 602 includes fluid passages 726 and 728 between corresponding openings 614 and 616 and fluid chamber 724, a fluid passage 730 between fluid chamber 724 and opening 620 of assay region 618, and fluid passages between each of openings 622, 624, and 626 of assay region 618 and a waste chamber 740. Waste chamber 740 may include an absorptive material to receive fluid from one or more fluid chambers of system 600. Body 602 may include a fluid passage 742 between waste chamber 740 and the outer surface of body 602, such as to release air displaced by fluid received within waste chamber 740.

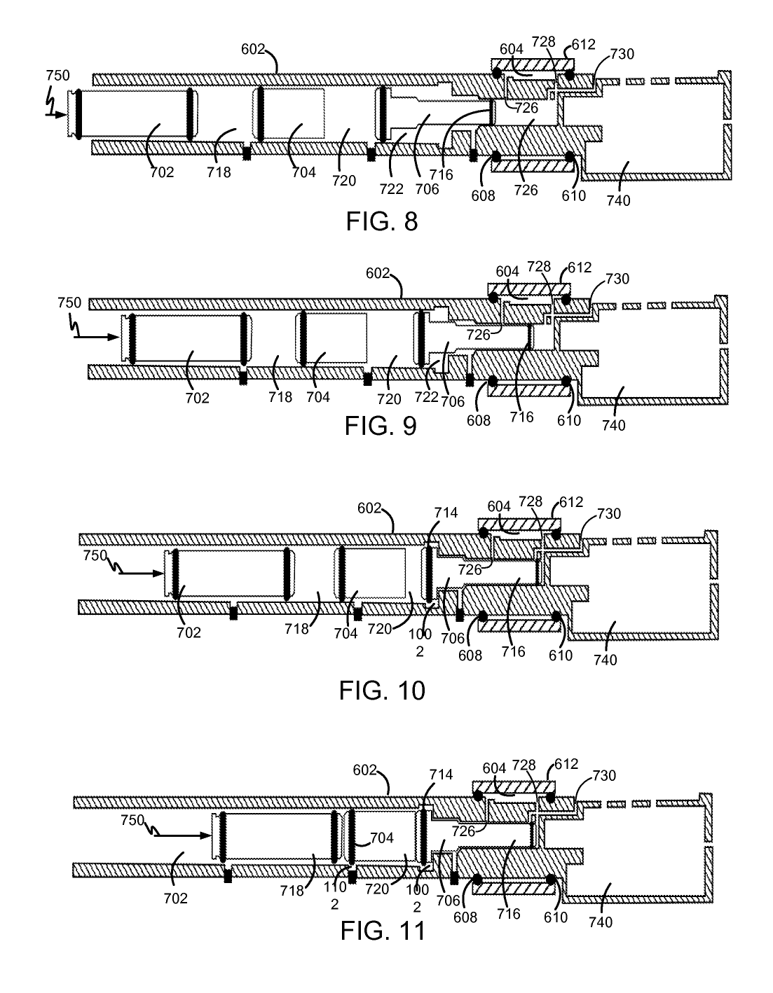

Body 602 may include one or more of fluid passages 744, 746, and 748 in fluid communication with corresponding fluid chambers 718, 720, and 722. One or more of fluid passages 744, 746, and 748 may have an opening through the outer surface of body 602, which may be used to provide one or more assay fluids to a corresponding fluid chamber during preparation procedure. Such an opening through the outer surface of body 602 may be plugged or sealed subsequent to the preparation procedure, such as illustrated in FIGS. 8-11. Alternatively, or additionally, one or more of fluid passages 744, 746, and 748 may include an opening to another fluid chamber of system 600, such as to provide a fluid bypass around one or more other fluid chambers and/or plungers.

Example operation of system 600 is described below with reference to FIGS. 8-14.

FIG. 8 is a cross-sectional view of system 600, wherein plungers 702, 704, and 706 are in corresponding initial or first positions.

FIG. 9 is a cross-sectional view of system 600, wherein plungers 702, 704, and 706 are in respective first intermediate positions.

FIG. 10 is a cross-sectional view of system 600, wherein plunger 704 is in a second position, and plungers 702 and 704 are in respective second intermediate positions.

FIG. 11 is a cross-sectional view of system 600, wherein plungers 702, 704 and 706 are in respective second positions.

FIGS. 8-11 may represent sequential positioning of plungers 702, 704 and 706 in response to a force in a direction 750 of FIG. 7.

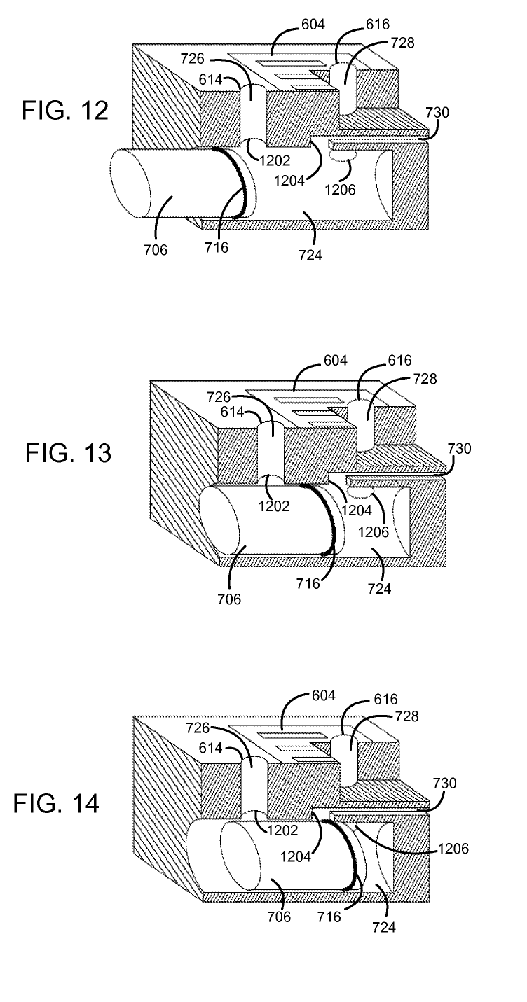

FIG. 12 is an expanded view of a portion of system 600, including a portion of plunger 706 in the first position corresponding to FIG. 8.

FIG. 13 is an expanded view of a of portion system 600, including a portion of plunger 706 in the intermediate position corresponding to FIG. 9, and including fluid directional arrows.

FIG. 14 is an expanded view of a portion of system 600, including a portion of plunger 706 in the second position corresponding to FIGS. 10 and 11.

During a preparation process, fluid chambers 718, 720, and 722, may be provided with corresponding first, second, and third fluids, and fluid chamber 724 may provided with a gas, such as air. The fluids in one or more of fluid chambers 718, 720, and 722 may be relatively incompressible compared with the gas in fluid chamber 724.

In FIG. 8, when the force is applied to plunger 702 in direction 750, the relatively incompressibility of the fluids in fluid chambers 718 and 720 transfer the force to plunger 706. Plungers 702, 704, and 706 may move together in direction 750.

As plungers 702, 704, and 706 move in direction 750, fluid within fluid chamber 724, which may include air, travels from fluid chamber 724, through fluid passage 730 to assay chamber 732, and through fluid passages 734, 736, and 738 to waste chamber 740.

Prior to O-ring 716 of plunger 706 passing an opening 1202 (FIG. 12) of fluid passage 726, fluid chamber 722 is substantially isolated and no fluid flows from fluid chamber 722 to fluid channel 728 or from fluid chamber 722 to fluid chamber 724.

As O-ring 716 of plunger 706 moves towards opening 1202, and as fluid chamber 722 is correspondingly moved in direction 750 into a narrower-diameter inner surface portion of body 602, a volume of fluid chamber 722 decreases. The reduced volume of fluid chamber 722 may increase a pressure of the fluid within fluid chamber 722. The fluid within fluid chamber 722 may include a combination of a relatively incompressible fluid and relatively compressible fluid, such as air, which may compress in response to the increased pressure.

In FIG. 9, when O-ring 716 is positioned between opening 1202 of fluid passage 726 and an opening 1204 of fluid passage 730, fluid chamber 722 is in fluid communication with fluid channel 726, while O-ring 716 precludes fluid flow directly between fluid chambers 722 and 724. The fluid in fluid chamber 722 may thus travel from fluid chamber 722, through fluid passage 726 to sample collection region 604, through fluid passage 728 to fluid chamber 724, through fluid passage fluid passage 730 to assay region 618, and through openings 722, 724, and 726 to waste chamber 740.

The fluid from fluid chamber 722 may contact and dislodge at least a portion of a sample contained within a sample pad 606, and may carry the sample to assay region 618, where the sample may react with a test membrane.

In FIG. 10, as plunger 706 reaches the second position and O-ring 716 passes opening 1204, a recess 1002 within an inner surface of body 602 provides a fluid passage around O-ring 714. Fluid within fluid chamber 720 travels through recess 1002, alongside plunger 706, through fluid passage 730 to assay chamber 732, and through fluid passages 734, 736, and 738 to waste chamber 740.

In FIG. 11, as plunger 704 reaches the second position, a recess 1102 within an inner surface of body 602 provides a fluid passage around O-ring 712 of plunger 704. Recess 1102 may correspond to fluid channel 746 in FIG. 7. Fluid within fluid chamber 718 travels through recess 1102, alongside plunger 704, through recess 102, alongside plunger 706, through fluid passage 730 to assay chamber 732, and through fluid passages 734, 736, and 738 to waste chamber 740.

As illustrated in FIG. 14, when plunger 706 is in the second position, O-ring 716 may be positioned between an opening 1402 of fluid channel 728 and an opening 1404 of fluid channel 730 to preclude fluid flow from sample collection region 604 to assay chamber 732 through fluid channels 728 and 730. This may be useful, for example, where the fluids within fluid chamber 720 and 718 are to contact an assay membrane within assay chamber 732 rather than sample pad 606 within sample collection region 604. This may be useful, for example, where the fluids within fluid chamber 720 and 718 include a wash fluid and/or a reactive material to wash and/or react with the assay membrane.

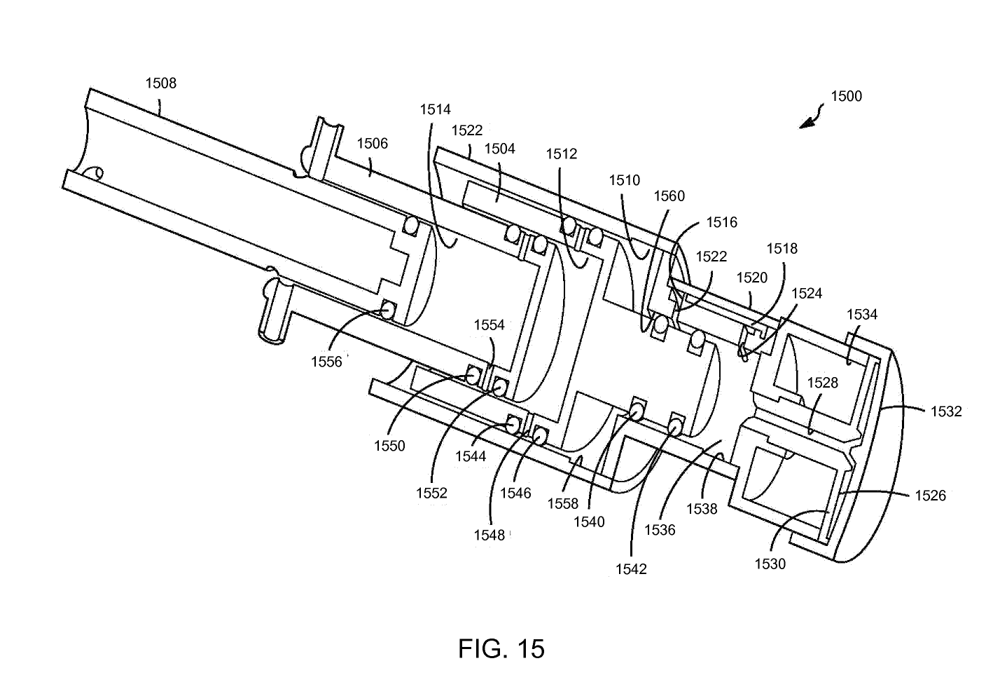

FIG. 15 is a cross-sectional perspective view of a portion of an assay system 1500 including a housing portion 1502 and a fluid controller system, including a plurality of fluid controllers, or plungers 1504, 1506, and 1508. Fluid controllers 1504, 1506, and 1508 define a plurality of fluid chambers, illustrated here as first, second, and third fluid chambers 1510, 1512, and 1514, respectively. Fluid controllers 1504, 1506, and 1508 are slideably nested within one another.

Housing portion 1502 includes a sample chamber 1516 to receive a sample, and may include a sample substrate, membrane or pad 1518. Housing portion 1502 may include a cover mechanism such as a cover portion 1520, which may be removable or hingedly coupled to housing portion 1502, as described above with respect to FIG. 3. Housing portion 1502 includes a sample chamber inlet 1522 and a sample chamber outlet 1524.

Housing portion 1502 includes an assay chamber 1526 and an assay chamber inlet 1528, and may include an assay substrate, membrane or pad 1528 to capture, react, and/or display assay results.

Housing portion 1502 includes an assay result viewer, illustrated here as a display window 1532 disposed over assay chamber 1528.

Housing portion 1502 includes a waste fluid chamber 1534 to receive fluids from assay chamber 1526.

Housing portion 1502 includes a transient fluid chamber 1536 having one or more fluid channels 1538, also referred to herein as a fluid controller bypass channel.

Housing portion 1502 further includes one or more other fluid channels 1558.

First fluid chamber 1510 includes a fluid chamber outlet 1560, illustrated here as a space between fluid controller 1506 and an inner surface of hosing portion 1502.

Second fluid chamber 1512 includes a fluid chamber outlet 1548, illustrated here as a gate or passage through fluid controller 1504.

Third fluid chamber 1514 includes a fluid chamber outlet 1554, illustrated here as a gate through fluid controller 1506.

Fluid controllers 1504, 1506, and 1508 include one or more sealing mechanisms, illustrated here as O-rings 1540 and 1542, O-rings 1544 and 1546, O-rings 1550 and 1552, and O-ring 1556.

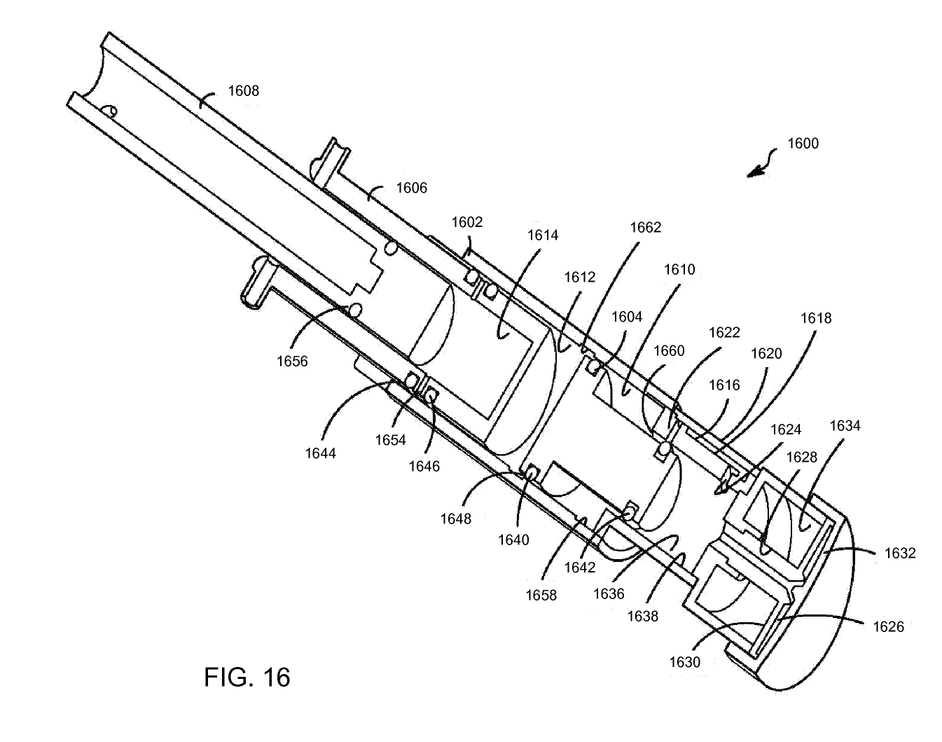

FIG. 16 is a cross-sectional perspective view of a portion of an assay system 1600 including a housing portion 1602 and a fluid controller system, including a plurality of fluid controllers, or plungers 1604, 1606, and 1608. Fluid controllers 1604, 1606, and 1608 define a plurality of fluid chambers, illustrated here as first, second, and third fluid chambers 1610, 1612, and 1614, respectively. Fluid controller 1608 is slideably nested within fluid controller 1606.

Housing portion 1602 includes a sample chamber 1616 to receive a sample, and may include a sample substrate 1618, which may include a surface of sample chamber 1616 or membrane therein. Housing portion 1602 may include a cover mechanism such as a cover portion 1620, which may be removable or hingedly coupled to housing portion 1602, as described above with respect to FIG. 3. Housing portion 1602 includes a sample chamber inlet 1622 and a sample chamber outlet 1624.

Housing portion 1602 includes an assay chamber 1626 and an assay chamber inlet 1628, and may include an assay substrate 1628 to capture, react, and/or display assay results. Assay substrate may include a surface of assay chamber 1626 or a membrane therein.

Housing portion 1602 includes an assay result viewer, illustrated here as a display window 1632 disposed over assay chamber 1628.

Housing portion 1602 includes a waste fluid chamber 1634 to receive fluids from assay chamber 1626.

Housing portion 1602 includes a transient fluid chamber 1636 having one or more fluid channels 1638, also referred to herein as a fluid controller bypass channel.

Housing portion 1602 further includes fluid channels 1658 and 1662.

First fluid chamber 1610 includes a fluid chamber outlet 1660, illustrated here as a space between fluid controller 1606 and an inner surface of hosing portion 1602.

Second fluid chamber 1612 includes a fluid chamber outlet 1648, illustrated here as a space between fluid controller 1604 and an inner surface of hosing portion 1602.

Third fluid chamber 1614 includes a fluid chamber outlet 1654, illustrated here as a gate or passage through fluid controller 1606.

Fluid controllers 1604, 1606, and 1608 include one or more sealing mechanisms, illustrated here as O-rings 1640 and 1642, O-rings 1644 and 1646, and O-ring 1656.

One or more inlets, outlets, openings, channels, and fluid pathways as described herein may be implemented as one or more of gates and passageways as described in one or more preceding examples, an may include one or more of:
a fluid channel within an inner surface of a housing;
a fluid passage within a housing, having a plurality of openings through an inner surface of the housing;
the fluid passage through a fluid controller; and
a fluid channel formed within an outer surface of one of the fluid controllers.

One or more inlets, outlets, openings, channels, fluid paths, gates, and passageways, as described herein, may include one or more flow restrictors, such as check valves, which may include a frangible check valve, to inhibit fluid flow when a pressure difference across the flow restrictor valve is below a threshold.

In FIG. 2, user-initiated actuator 204 may include one or more of a mechanical actuator, an electrical actuator, an electro-mechanical actuator, and a chemical reaction initiated actuator. Example user-initiated actuator systems are disclosed below, which may be implemented with pumps disclosed above.

An assay system as disclosed herein may include a user-rupturable membrane to separate a plurality of chemicals within a flexible tear-resistant membrane. The chemicals may be selected such that, when combined, a pressurized fluid is generated. The pressurized fluid may be gas or liquid. The pressurized fluid may cause fluid controllers to move as described in one or more examples above. Multiple user-rupturable membranes may be implemented for multiple fluid passages.

Methods and systems to activate assay systems are disclosed below.

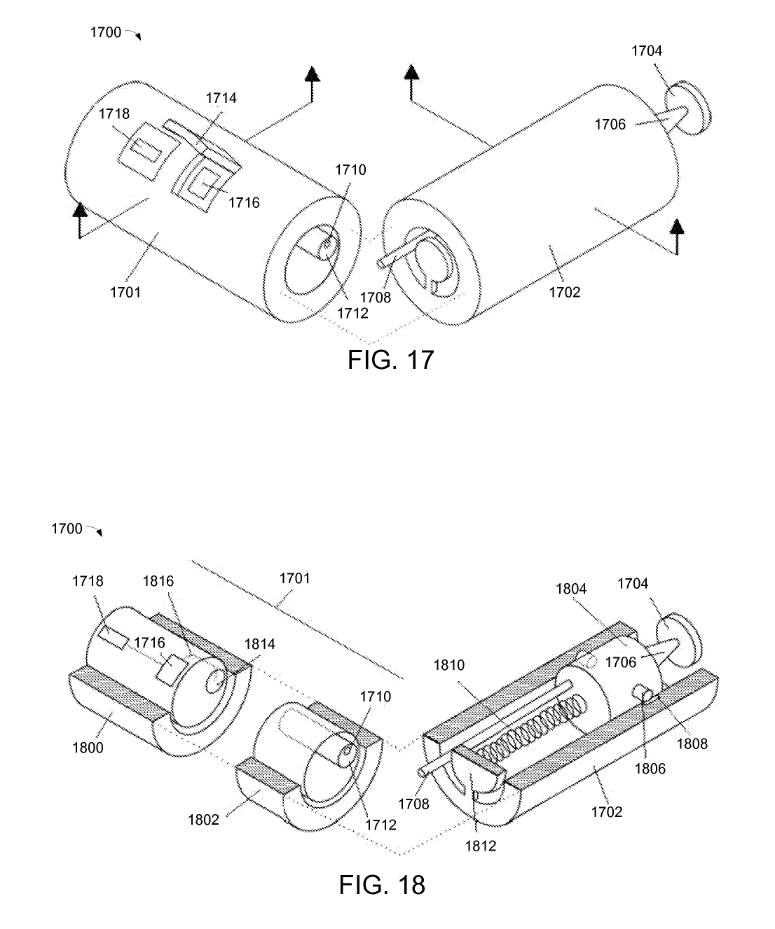

FIG. 17 is a profile view of a fluidic assay system 1700 including first and second portions or sections 1701 and 1702.

FIG. 18 is a cross-sectional view of system 1700, including elements 1800 and 1802 of portion 1701.

Section 1702 may include a user initiated thumb press, pushbutton, or cap 1704 that is connected to a rotational post, rod or axle 1706 that is affixed to a control device 1804 which may have directional control post 1806 that is contained in an internal track 1808. Rotational post 1706 allows control device 1804 to rotate independently of the thumb press 1704.

Section 1802 may include one or more fluid chambers, cavities, and/or voids 1712 that are activated by the rod 1708 connected to the control device 1804. The fluid chambers may contain plungers separating fluid, fluid under pressure, or fluid packages that burst when activated at point 1710 by rod 1708.

Section 1701 may contain fluid pathways, channels, or ducts 1816 that connect to one or more of a sample collection region 1716 and a sample analyzing or assay region 1718, which may include an assay result observation region. Sample collection region 1716 may have a glass fiber pad or other material that can absorb a sample and a cover or window 1714 to seal the chamber to prevent additional sample from being applied.

Sample analyzing region 1718 may have a test membrane, such as nitrous cellulose, with immobilized antigens or other substance to detect the presence of certain analytes in the sample.

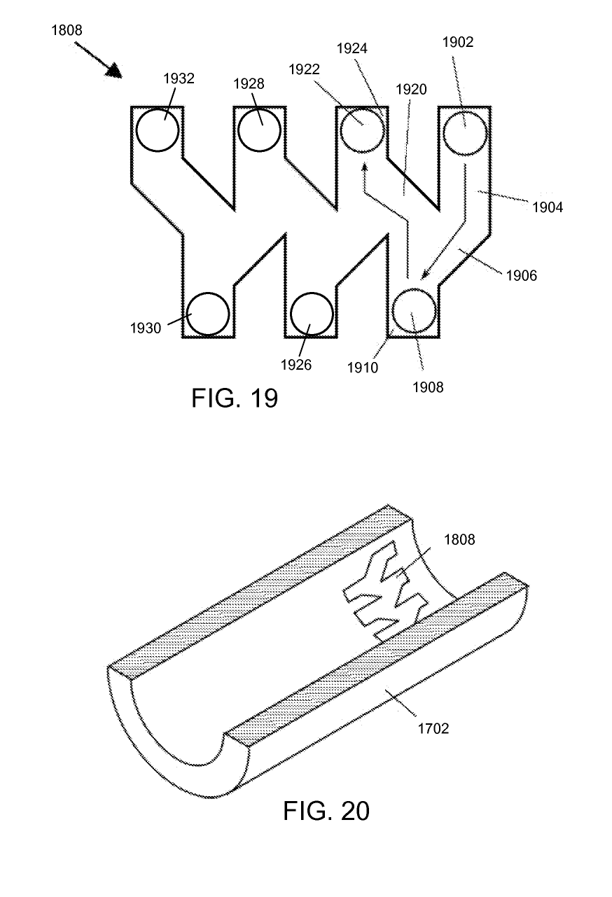

Operation of control post 1806 is described below with reference to FIGS. 19 and 20. FIG. 19 is a graphic depiction of an example pathway of internal track 1808 to guide control post 1806. FIG. 20 is corresponding a cross-sectional view of portion 1702, including an internal track 1808 formed within an inner surface thereof, corresponding to the pathway of FIG. 19. Internal track 1808 is not, however, limited to the examples of FIGS. 19 and 20.

In FIG. 19, control post 1806 slidingly engages track 1808 beginning at a position 1902, such as in response to a force applied to thumb press 1704 in FIG. 17, and in response to a force applied by spring 1810.

In response to a force applied to thumb press 1704 in a direction 1950, control post 2806 travels from position 1902, along a linear portion 1904 of track 1808, through an angled portion 1906, and into a position 1908. Position 1908 may correspond to a depressed position of thumb press 1704 and a corresponding compressed position of spring 1810.

As control post 1806 travels from position 1902 to position 1908, control device 1804 moves linearly relative to portion 1702, in direction 1950.

As control post 1806 travels along angled portion 1906, a directional or rotational force is imparted to control post 1806 by a side wall of angled portion 1906, to cause control device 1804 to rotate relative to portion 1702. A rotational distance of control unit 1804 may correspond to a shortest distance between portions 1906 and 1910.

Upon release of thumb press 1704, compressed spring 1810 applies a force to control post 1806 in a direction 1952, to cause control post 1806 to travel into an angled portion 1920 of path 1802, which guides control post 1806 into a position 1922 of a portion 1924.

As control post 1806 travels from position 1908 to position 1922, control device 1804 moves linearly relative to portion 1702, in direction 1952.

As control post 1806 travels along angled portion 1920, a directional or rotational force is imparted to control post 1806, such as by a side wall of angled portion 1920, to cause control device 1804 to further rotate relative to portion 1702. A rotational distance of control unit 1804 may correspond to a shortest distance between portions 1910 and 1924.

Thumb press 1704 may be repeatedly depressed and released to move control post 1806 along additional portions of track 1808, such as to positions 1926 through 1932.

Linear and/or rotation movement of control device 1804 relative to portion 1702 may control and/or align elements of system 1700 to activate and/or control one or more assay features. For example, linear and rotational movement of control device 1804 may position control rod 1708 in FIGS. 17 and 18 relative to one or more activation points associated with portion 1701.

For example, control rod 1708 may control movement of a plunger and/or other elements within portion 1701, relative to one or more other elements within portion 1701. Alternatively, or additionally, rod 1708 may include a fluid passage to permit fluid to flow between fluid chamber 1712 and another fluid chamber and/or fluid passage via rod 1708.

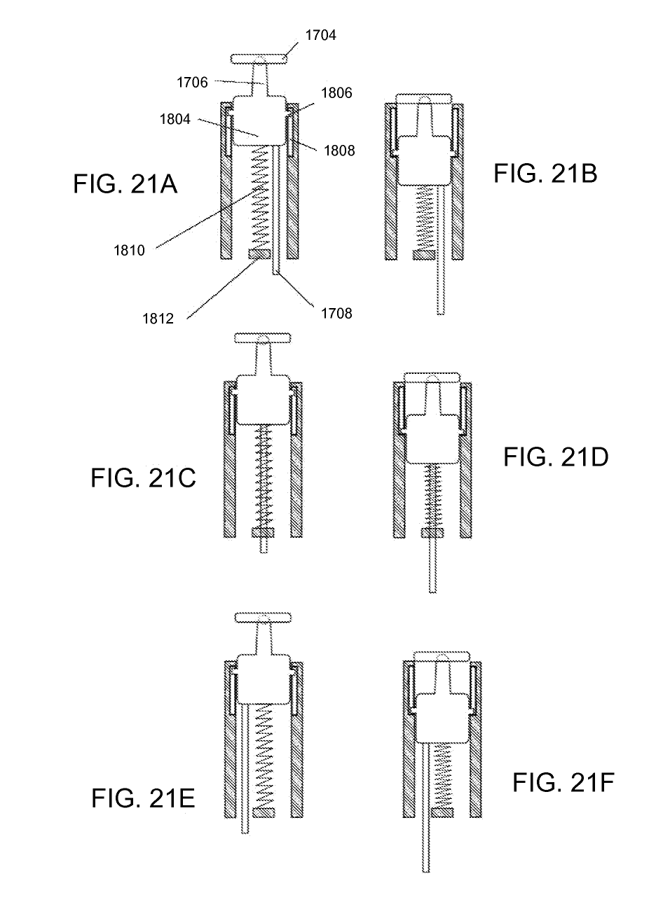

FIGS. 21A through 21F are cross-sectional views of portion 1702, depicting an example sequence of movements of in response to repeated pressing and releasing of thumb press 104. FIGS. 21A through 21F may correspond to positions 1902, 1908, 1922, 1926, 1928, and 1930, respectively, in FIG. 19. Each of the positions associated with FIGS. 21B through 21F may be associated with a corresponding assay activity. Alternatively, one or more of the positions associated with FIGS. 21B through 21F may correspond to an interim or transitory position, such as to provide a reaction time.

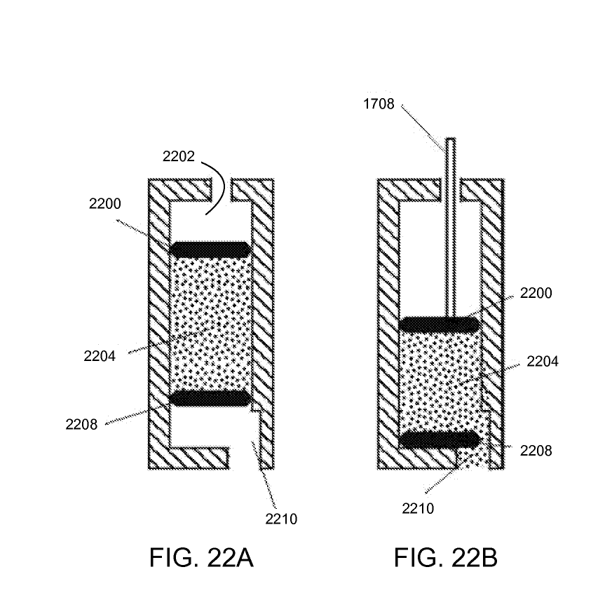

In FIG. 18, section 1802 may include one or more fluid chambers 1712 that are activated by the rod 1708 at point 1710. FIGS. 22A and 22B are cross-sectional views of a fluid chamber 2202, including first and second plungers 2200 and 2208.

In FIG. 22A, rod 1708 pushes down on first plunger 2200, which transfers force through fluid 2204 to second plunger 2208.

In FIG. 22B, the force applied to second plunger 2208 causes second plunger 2208 to move to an open position defined with respect to a bypass channel 2210, to release at least a portion of fluid 2204 from fluid chamber 2202.

Fluid exiting fluid chamber 2202 may enter a region 1814 in FIG. 18, which may be aligned with a fluid passage 1816. One or more other fluid chambers may also include fluid exit passages aligned with corresponding fluid passages 1816. One or more of the fluid passages 1816 may connect to sample collection region 1716 and/or to an assay reaction region 1718. Fluid channels that connect to the sample collection region 1716 may bring the sample to the assay reaction region 1718.

An assay apparatus as disclosed herein may be implemented to include all or substantially all components needed to carry out a test, such as an immunoassay, which may provide portability and which may permit operation relatively little training

An assay apparatus as disclosed herein may be implemented to activate fluid chambers in a specific order, which may reduce the possibility of operator error.

An assay apparatus as disclosed herein may be implemented to provide a reaction time between pressings and/or releasing of thumb press 104. Such reaction time(s) may be controlled by a timer device and/or by an operator.

An assay apparatus as disclosed herein may be reconfigurable for different fluid components and/or assays. For example, a plurality of portions 1701 may each be configured differently from one another, and portion 1702 may be configured to couple to each of the a plurality of portions 1701.

Portions of an assay apparatus as disclosed herein may be re-usable. For example, the sample collection and test section may be replaceable and disposable.

While various embodiments are disclosed herein, it should be understood that they have been presented by way of example only, and not limitation. It will be apparent to persons skilled in the relevant art that various changes in form and detail may be made therein without departing from the spirit and scope of the methods and systems disclosed herein. Thus, the breadth and scope of the claims should not be limited by any of the example embodiments disclosed herein.

What is claimed is:

1. A portable, point-of-care, self contained assay system, comprising:
a portable housing having a sample collection region, a sample analyzing region, a plurality of fluid chambers, and a plurality of fluid passages from the fluid chambers to one or more of the sample collection region and the sample analyzing region, wherein fluid outlets of the fluid chambers are movable relative to the fluid passages; and
a mechanically actuated fluid controller to repeatedly reposition the fluid chamber fluid outlets relative to the fluid passages and to sequentially force fluid from each of a plurality of sets of one or more of the fluid chambers, through corresponding fluid chamber outlets, and to one or more of the sample collection region and the sample analyzing region subsequent to at least a subset of the repositionings.

2. The system of claim 1, wherein the mechanically actuated fluid controller is configured to laterally and rotationally reposition the fluid chamber outlets relative to the fluid passages.

3. The system of claim 1, wherein the mechanically actuated fluid controller is configured to laterally and rotationally reposition the fluid chamber outlets relative to the fluid passages in accordance with a sequence of predetermined positions, each including a lateral component and a rotational component.

4. The system of claim 3, wherein each of the predetermined positions includes one of a sequence of rotational positions and an alternating one of a first and a second lateral position.

5. The system of claim 3, wherein the mechanically actuated fluid controller includes:
a control device movable within the portable housing to control movement of the fluid chamber fluid outlets relative to the fluid passages; and
a mechanical actuator;
wherein the control device is configured to sequentially move through the sequence of predetermined positions in response to movement of the mechanical actuator and in accordance with a mechanical pattern imbedded within the system.

6. The system of claim 5, wherein:
the mechanical pattern includes a recessed path within an inner surface of the portable housing;
the control device includes a post extending from the control device to slideably engage the recessed path; and
the recessed path is patterned to guide the control device through the sequence of predetermined positions in response to movement of the mechanical actuator.

7. The system of claim 6, wherein:
the mechanical actuator includes a button extending through a surface of the portable housing; and
the recessed path is patterned to guide the control device through the sequence of predetermined positions in response to lateral movement of the button.

8. The system of claim 7, wherein:
the button is configured to move the control device from a first lateral position to a second lateral position in response to movement of the button from a first position to a second position; and
a compression device to compress in response to movement of the button from the first position to the second position, and to move the control device from the second lateral position to the first lateral position and the button from the second position to the first position when the compression device is compressed;
wherein each of the predetermined positions includes one of a sequence of rotational positions and an alternating one of the first and second lateral positions.

9. The system of claim 8, further including:
a coupling device to restrain the compression device relative to the control device while the button is pressed, and to thereafter release the compression device to move the control device from the second lateral position to the first lateral position and the button from the second position to the first position.

10. The system of claim 6, wherein:
the mechanical actuator includes a mechanically twistable device to rotate the control device relative to the portable housing; and
the recessed path includes a spiral pattern to guide the control device through the sequence of predetermined positions in response to rotational movement of the twistable device, including to rotationally and laterally move the control device relative to the portable housing.

11. The system of claim 1, wherein the mechanically actuated fluid controller includes:
a control device movable within the portable housing to control movement of the fluid chamber fluid outlets relative to the fluid passages; and
a post extending from a the control device to slideably engage a recessed path of an inner surface of the portable housing;
wherein the recessed path is patterned to guide the control device through a sequence of predetermined positions in response to movement of the control device.

12. The system of claim 11, wherein the control device includes a rod extending therefrom to impart the sequence of predetermined positions to the fluid chamber fluid outlets relative to the fluid passages.

13. The system of claim 11, wherein:
the control device is laterally and rotationally movable within the portable housing; and
the recessed path is patterned to guide the control device through the sequence of predetermined positions, each including a lateral component and a rotational component.

14. The system of claim 13, wherein each of the predetermined positions includes one of a sequence of rotational components and an alternating one of a first and a second lateral component.

15. The system of claim 11, wherein the control device includes:
a mechanical button extending through a surface of the portable housing to laterally move the control device.

16. The system of claim 11, wherein the control device includes:
a mechanically twistable device to rotate the control device relative to the portable housing.

17. A method of operating a portable, point-of-care, self contained assay system that includes a portable housing having a sample collection region, a sample analyzing region, a plurality of fluid chambers, and a plurality of fluid passages to one or more of the sample collection region and the reaction region, wherein fluid outlets of the fluid chambers are movable relative to the fluid passages, the method comprising:
repeatedly repositioning the fluid chamber fluid outlets relative to the fluid passages in response to lateral movement of a button extending from the assay system housing; and
sequentially forcing fluid from each of a plurality of sets of one or more of the fluid chambers, through corresponding fluid chamber outlets, and to one or more of the sample collection region and the sample analyzing region subsequent to at least a subset of the repositionings.

18. The method of claim 17, wherein the repositioning includes laterally and rotationally repositioning the fluid chamber outlets relative to the fluid passages.

19. The method of claim 17 wherein the repositioning includes laterally and rotationally repositioning the fluid chamber outlets relative to the fluid passages in accordance with a sequence of predetermined positions, each including a lateral component and a rotational component.

20. The method of claim 19, wherein each of the predetermined positions includes one of a sequence of rotational positions and an alternating one of a first and a second lateral position.

21. The method of claim 19, wherein the repositioning further includes:
sequentially repositioning through the sequence of predetermined positions in accordance with a mechanical pattern imbedded within the assay system.

22. The method of claim 21, wherein the mechanical pattern includes a recessed path within an inner surface of the portable housing and the assay system includes a fluid control device movable within the assay system housing and having a post extending therefrom to slideably engage the recessed path, and wherein the repositioning further includes;
guiding the post through the recessed path to move the control device through the sequence of predetermined positions in response to movement of the fluid control device relative to the assay system housing.

23. The method of claim 22, wherein the repositioning further includes:
repositioning in response to a mechanically operated button coupled to the fluid control device.

24. The method of claim 22, wherein the repositioning further includes:
repositioning in response to a mechanically operated rotational device coupled to the fluid control device.
